(19) United States
(12) Patent Application Publication
(10) Pub. No.: US 2018/0168929 A1
(43) Pub. Date: Jun. 21, 2018

(54) VIAL WITH INTERGRATED NEEDLESS ACCESS PORT

(71) Applicants: Jason Stone, Orlando, FL (US); Julian Quinones, Orlando, FL (US)

(72) Inventors: Jason Stone, Orlando, FL (US); Julian Quinones, Orlando, FL (US)

(21) Appl. No.: 15/387,014

(22) Filed: Dec. 21, 2016

(51) Int. Cl. A61J 1/14; A61J 1/06; A61J 1/22; A61M 5/31

(52) U.S. Cl. CPC A61J 1/1481; A61M 5/3129; A61J 1/22; A61J 1/065

(57) ABSTRACT

A needleless access vial includes a vial body for storing a liquid medication. A needleless access port is disposed within an opening along the top of the vial body. A valve having a deformable body is positioned within the access port housing, and is in communication with the access port inlet section and the access port outlet section. The valve is biased toward a closed configuration, and transitions to an open configuration upon engagement of the outlet section by a needleless syringe.

### TECHNICAL FIELD

The present invention relates generally to medical devices, and more particularly to a medicinal vial with an integrated needless access port.

### BACKGROUND

The statements in this section merely provide background information related to the present disclosure and may not constitute prior art.

Liquid medication intended for parenteral administration is typically provided within a single-dose glass vial that is sealed along a top opening by a rubber bung. In order for a health care provider to access the medication, they must first secure a needle onto the end of a syringe, and then pierce the bung with the needle to draw the medication out of the vial.

Owing to strict standards governing safe injection practices, health care providers are taught to utilize one needle, one syringe, only one time. As such, upon removing the medication from the vial, the healthcare provider must remove and dispose the used needle, before attaching a second needle to the syringe, and injecting the patient.

In situations where seconds can mean the difference between life and death, such a procedure utilizes precious time for which a patient may not have. Moreover, the utilization of so many needles imposes a significant risk to the health care providers themselves, especially paramedics who must perform this procedure within a moving vehicle. To this end, the Centers for Disease Control and Prevention (CDC) estimates that about 385,000 sharps-related injuries occur annually among health care workers throughout the United States.

The present invention, directed to a medicinal vial with an integrated needless access port differs from the conventional art in a number of aspects. The manner by which will become more apparent in the description which follows, particularly when read in conjunction with the accompanying drawings.

### SUMMARY OF THE INVENTION

The present invention is directed to a needleless access vial. One embodiment of the present invention can include a medical vial having a shape and size that is suitable for storing liquid medication. Projecting from the top end of the vial is a needleless access port having an internal valve for accessing the liquid medication.

The valve is preferably biased toward a closed configuration, and can be moved to an open configuration upon being accessed by a needleless syringe, to allow the liquid medication to pass from the vial to the syringe.

Another embodiment of the present invention can include the access port being permanently and hermetically sealed to the vial.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of the vial with integrated needless access port, that is useful for understanding the inventive concepts disclosed herein.

FIG. 2A is a perspective view of the needleless access port of the vial, in accordance with one embodiment of the invention.

FIG. 2B is a cross-sectional cutout view of the needleless access port of the vial, in accordance with one embodiment of the invention.

FIG. 3 is a cross-sectional cutout view of the vial with integrated needless access port in operation, in accordance with one embodiment of the invention.

### DETAILED DESCRIPTION OF THE INVENTION

As described herein, the term “removably secured,” and derivatives thereof shall be used to describe a situation wherein two or more objects are joined together in a non-permanent manner so as to allow the same objects to be repeatedly joined and separated.

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

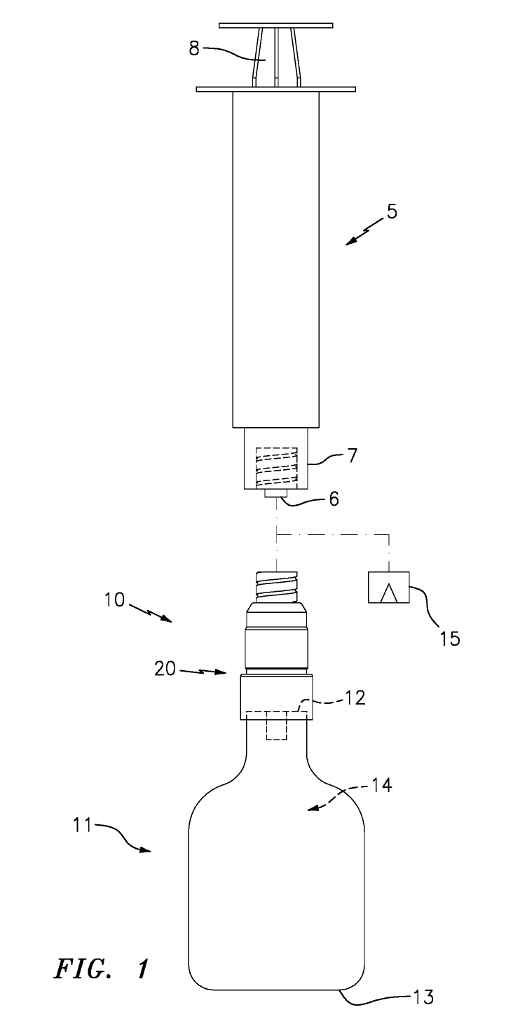

FIGS. 1-3 illustrate one embodiment of a vial with an integrated needless access port 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

As shown in FIG. 1, the vial 10 can include a main body 11 for receiving and storing liquid medications, a tamper-evident seal 15, and a needleless access port 20 that is adapted for use with a needleless device such as a syringe 5.

As described herein, the tamper-evident seal 15 can include any number of different capping devices capable of providing a clear indication that the contents of the vial have been opened or otherwise accessed. The seal 15 can include, for example, single use plastic caps and/or adhesive-backed sheets, as are commonly utilized for such purpose. The seal 15 can be removably secured to the top end of the below described access port and must be removed before the same can be accessed by the needleless device.

As described herein, the main body 11 can preferably include a conventional structure in the form of a single or multi-dose glass vial having an opening 12 along the top end thereof, and a generally flat bottom end 13 that define a hollow interior space 14. Of course, the main body is not limited to the use of glass, as any number of other materials such as plastic, for example, that are suitable for storing liquid medications are also contemplated.

The needleless access port 20 can preferably include a positive pressure valve that is hermetically sealed along the opening 12 of the main body 11 via a cap 29, so as to form a permanent, airtight connection with the interior space 14 at the time of construction. In this regard, the opening 12 may also include a lip (not illustrated) or other type of ledge onto which the cap 29 can engage.

As will be described below, the access port 20 can be used in conjunction with a conventional syringe 5 having a male luer lock that typically comprises a centrally located tip 6, and an internally threaded shield 7 surrounding the tip 6 to remove the liquid medication stored within the vial without requiring the use of a needle. One example of a needleless access port 20 which can be utilized herein is described in U.S. Pat. No. 5,782,816, to Werschmidt, the contents of which are incorporated by reference in its entirety.

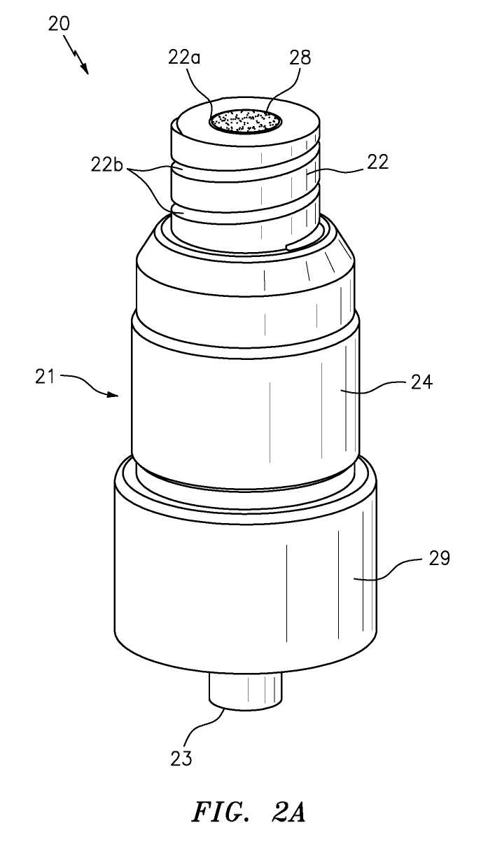

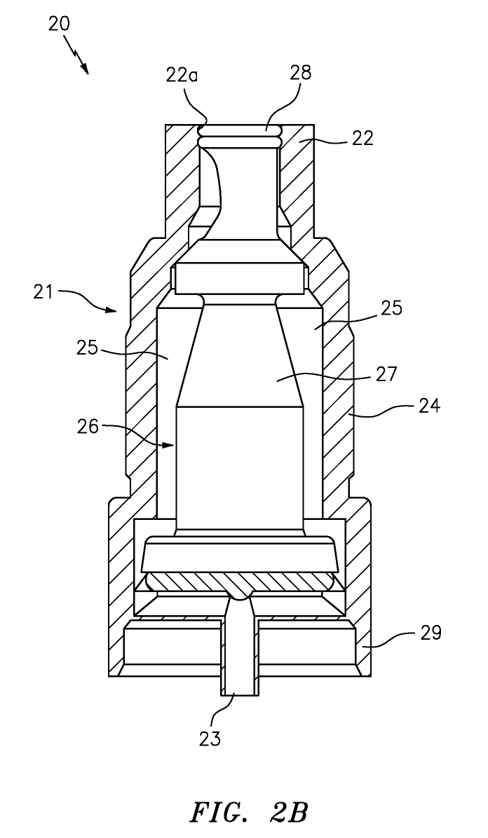

As shown in FIGS. 2A and 2B, the access port 20 can include a housing 21 having an outlet 22, an inlet 23 and a central housing section 24, that define a fluid passageway 25 for removing the liquid medication from the hollow interior space 14 of the vial. A valve 26 is disposed within the housing 21, and includes a generally resilient and deformable body 27 and a tip member 28. The tip 28 is flush with the opening 22a of the outlet 22 such that a deformable seal is defined and formed to prevent contamination of the liquid medication though the access port 20.

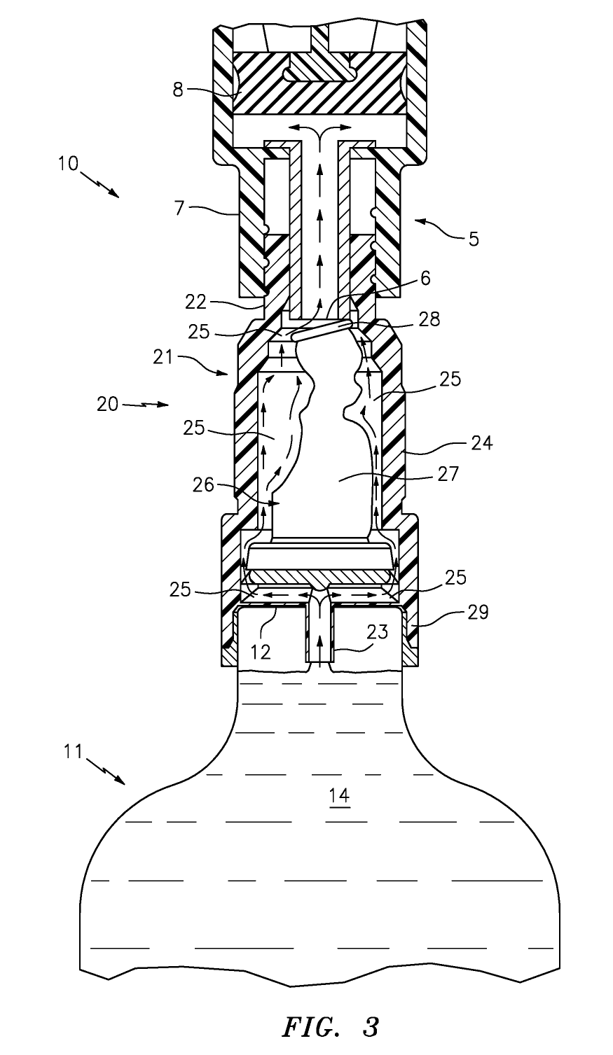

The valve 26 is biased to a closed configuration (See FIG. 2B) in a resting state, so as to prevent any contaminants from making contact with the liquid medication via the passageway 25. However, the valve 26 can be switched to an open configuration (See FIG. 3) through contact with a needleless syringe 5, to allow the medication stored within the vial to be pulled into the syringe via the fluid passageway 25. Owing to the resilient nature of the valve body 27 and tip 28, the valve can function to maintain an airtight seal with the outlet 22 of the housing 21 regardless of the number of times the same is accessed by a syringe.

FIG. 3 illustrates one embodiment of the device 10 in operation. As shown, the tip 6, of the needleless syringe 5 can be positioned against the tip of the valve 28 located within the outlet 22 of the access port 20. When so positioned, the threads of the internally threaded shield 7 and the complementary threaded elements 22b along the outlet 22 can be engaged via a pushing and twisting motion that removably secures the syringe to the port. Because the axial length of the tip 6 is greater than the axial length of the threaded shield 7, this motion causes the tip 6 to push the tip of the valve 28 inwards, thereby positioning the valve to the open configuration.

When in the open configuration, the plunger 8 of the syringe 5 can be pulled outward, so as to create a vacuum effect that sucks the liquid medication stored within the vial 11 through the access port 20 as a fluid passageway 25. Upon removing the desired amount of medication, the syringe can be separated from the access port via a twisting motion in the opposite direction, at which time the valve 26 will return to the closed configuration, and the syringe 5 can be utilized to administer the medication.

Although described with respect to particular shapes, sizes and/or types of fittings, this is for illustrative purposes only, as the access port 20 can include any number of different shapes, sizes and/or types of fittings suitable for engaging any type of needleless device. Moreover, although the needleless device described herein has been in the form of a syringe, this is also for illustrative purposes. As such, the vial 10 can be configured for use with any type of medical device having an adapter and/or connector for receiving and/or dispensing liquid medication.

As described herein, one or more elements of the vial with integrated needless access port 10 can be secured together utilizing any number of known attachment means such as, for example, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

Accordingly, the vial with integrated needless access port provides a novel solution to providing quick access to liquid medication without the use of needles and eliminating the associated risks to healthcare workers.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the terms “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A medicinal vial for dispensing liquid medication to a needleless syringe having a tip and a threaded shield, said comprising:
a vial body having a bottom end, a continuous outer wall, a hollow interior space, and an opening that is in communication with the hollow interior space;
an access port housing that includes an inlet section disposed within the hollow interior space, an outlet section that is disposed above the opening, and a cap that is permanently sealed along the opening; and
a valve that is positioned within the access port housing, and is in communication with each of the inlet section and the outlet section, said valve functioning to transition between an open configuration for allowing access to the hollow interior space, and a closed configuration for preventing access to the hollow interior space.

2. The vial of claim 1, further comprising:
a plurality of threaded elements that are disposed along an outside portion of the outlet section, said outlet section and said threaded elements including a shape and size that are configured to engage the threaded shield of the needleless syringe;

3. The vial of claim 2, wherein said valve comprises:
a deformable body having a tip member that is disposed within an opening along the outlet section,
wherein in the closed configuration, the tip member is flush with the opening forming a seal, and
in the open configuration, the tip of the needleless syringe is positioned within the opening of the outlet section, and the tip member of the valve does not form a seal.

4. The vial of claim 1, wherein the access port housing is hermetically sealed to the vial body.

5. The vial of claim 1, wherein the vial body comprises a single-dose glass vial for storage of liquid medication.
